(19) United States
(12) Patent Application Publication
(10) Pub. No.: US 2025/0326717 A1
(43) Pub. Date: Oct. 23, 2025

(54) METHOD FOR SYNTHESIS OF CHLORFENAPYR FROM TRALOPYRIL AND ETHYLAL

(71) Applicants: ANAND SAMIAPPAN, Karur (IN); VENKAT REDDY SINGIREDDY, Hyderabad (IN); SAMPATH KUMAR LAXMALLA, Medchal (IN)

(72) Inventors: ANAND SAMIAPPAN, Karur (IN); VENKAT REDDY SINGIREDDY, Hyderabad (IN); SAMPATH KUMAR LAXMALLA, Medchal (IN)

(21) Appl. No.: 18/640,053

(22) Filed: Apr. 19, 2024

(51) Int. Cl. C07D 207/34

(52) U.S. Cl. CPC C07D 207/34

(57) ABSTRACT

The present invention relates to a commercially viable method of preparation of Chlorfenapyr, which is a halogenated pyrrole insecticide.

The present invention particularly relates to the method of synthesis of Chlorfenapyr from tralopyril (also known as 4-bromo-2-(4-chlorophenyl)-5-(trifluoromethyl)-1H-pyrrole-3-carbonitrile) and ethylal (also known as diethoxymethane) in presence of thionyl chloride as halogenating agent in the yield exceeding 90% w/w and possessing high purity of greater than 98% w/w.

Chlorfenapyr is useful as insecticide for termite control and crop protection against a variety of insect and mite pests.

### FIELD OF THE INVENTION

The present invention relates to a commercially viable method of preparation of Chlorfenapyr, which is a halogenated pyrrole insecticide.

The present invention particularly relates to the method of synthesis of Chlorfenapyr from tralopyril (also known as 4-bromo-2-(4-chlorophenyl)-5-(trifluoromethyl)-1H-pyrrole-3-carbonitrile) and ethylal (also known as Diethoxymethane) in presence of thionyl chloride as halogenating agent in the yield exceeding 90% w/w and possessing high purity of greater than 98% w/w.

### BACKGROUND OF THE INVENTION

In general, heteroarylpyrrole compounds and their derivatives are known to be possessing potent insecticidal, acaricidal, nematocidal, and molluscicidal agents for both plants and animals. Chlorfenapyr is a heteroarylpyrrole compound which is an approved pro-insecticide that becomes active after entering the host and is derived from a class of microbiologically produced compounds known as halogenated pyrroles, specifically 2-arylpyrrole compounds.

Chlorfenapyr inhibits the synthesis of adenosine triphosphate by oxidative removal of the N-ethoxymethyl group from chlorfenapyr by mixed functional oxidases resulting into a compound with insecticidal activity that decouples oxidative phosphorylation in the mitochondria, leading to disruption of ATP production, cell death and ultimately the death of the organism.

Chlorfenapyr has a huge demand in the market due to its well known broad spectrum of insecticidal and acaricidal effects, low mammalian toxicity and high efficiency. At present, the conventional method of synthesis of Chlorfenapyr includes alkoxymethylation of pyrroles on nitrogen to form N-(alkoxymethyl) pyrrole achieved by the condensation of the appropriate pyrrole with an a-halomethyl ether in the presence of a strong base. However, due to the high carcinogenic characteristics of such ethers (a-halomethyl ether), their employment is undesirable on commercial scale. Various synthetic routes for the production of Chlorfenapyr have been reported.

Wang Zhenjiang et al. in CN114524758 disclosed a method for synthesizing chlorfenapyr, having application in the field of pesticides comprising steps of using acetonitrile as a solvent, adding p-chlorophenylglycine, trifluoroacetic acid, and triethylamine as an acid- binding agent, uniformly stirring, dropwise adding phosphorus trichloride to carry out acylation reaction, and after the reaction is finished, extracting, desolventizing, and removing an extracting agent.

Xie Jianwu et al. in CN104016899 disclosed a method of production of chlorfenapyr comprising of
1. Synthesizing the intermediate 4-(p-chlorophenyl)-2-trifluoromethyl-3-oxazolin-5-one;

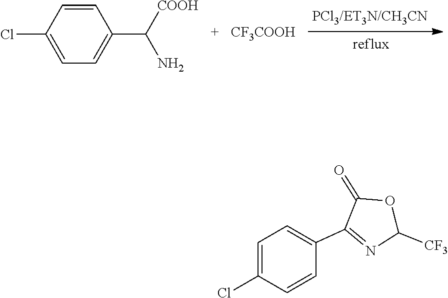 [SMILES: NC(C(=O)O)c1ccc(Cl)cc1 ; "CF3COOH" ; O=C1OC(C(F)(F)F)N=C1c1ccc(Cl)cc1]

2. Synthesizing the intermediate 2-(p-chlorophenyl)-5-trifluoromethyl-pyrrole-3-carbonitrile;

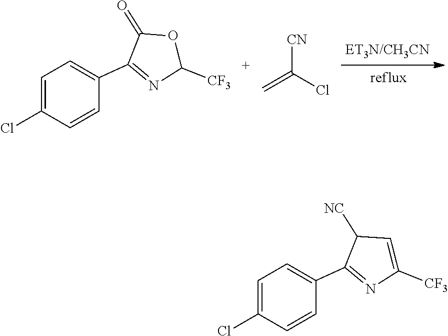

3. Synthesizing the intermediate 4-bromo-2-(p-chlorophenyl)-5-trifluoromethyl-pyrrole- 3-carbonitrile;

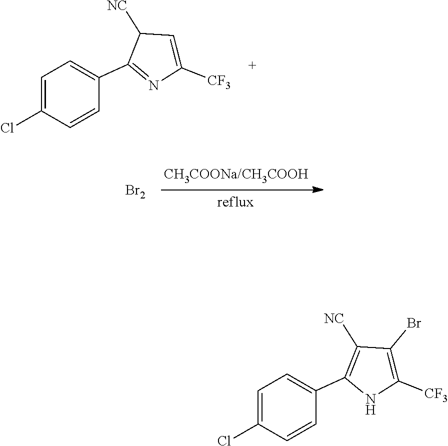 [SMILES: N#CC1C=C(C(F)(F)F)N=C1c1ccc(Cl)cc1 ; "Br2" ; N#Cc1c(-c2ccc(Cl)cc2)[nH]c(C(F)(F)F)c1Br]

4. Synthesizing the desired product Chlorfenapyr or 4-bromo-2-(p-chlorophenyl)-1-ethoxymethyl-5-trifluoromethyl-pyrrole-3-carbonitrile.

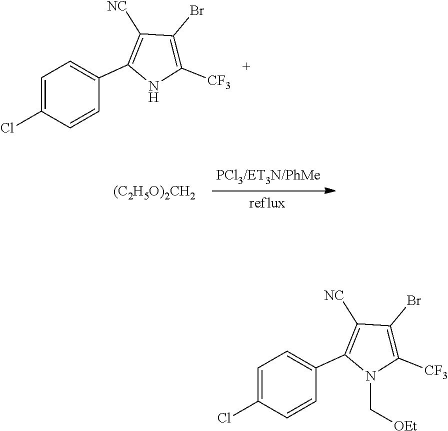 [SMILES: "(C2H5O)2CH2" ; N#Cc1c(-c2ccc(Cl)cc2)[nH]c(C(F)(F)F)c1Br ; CCOCn1c(-c2ccc(Cl)cc2)c(C#N)c(Br)c1C(F)(F)F]

Synthesis of the raw materials or starting materials for raw pesticides are abundantly reported in literature resources and their synthetic technology has mild reaction conditions and is suitable for industrial production.

Cortes David et al. in WO2018166819 disclosed a process for the production of arylpyrrol compounds using di (C₁-C₄-alkoxy) methane as one of the reactants and either POCl₃ (phosphorus oxychloride) or a mixture comprising POCl₃ (phosphorus oxychloride) and DMF (dimethylformamide), as reagents in the presence of di-isopropylethylamine (DIPEA).

Wang Zhenjiang et al. in CN110218170 disclosed a method for producing chlorfenapyr catalytically using a mixed catalyst. Material addition, chloric ether dropping, heat-preservation reactions and solid-liquid separation are all steps involved in the manufacturing process. The main raw materials used are 4-bromine and chloric ether and acetonitrile is used as a reaction solvent. Potassium carbonate and solid caustic soda are used as a mixed catalyst for catalytic synthesis of Chlorfenapyr, solid-liquid separation is performed after reactions are completed, a reaction solvent is evaporated off and a refining solvent is then used to carry out refining and thus Chlorfenapyr is prepared.

Hys, V. Y., Shevchuk, O. I., et al. in European Journal of Organic Chemistry (2020) disclosed the functionalization of 2-trifluoromethyl-1H-pyrrole: a convenient entry into advanced fluorinated building blocks including all isomeric 2-(trifluoromethyl) pralines. It disclosed synthetic utility of 2-trifluoromethyl-1H-pyrrole as a pharmaceutically relevant platform by the preparation of mono-and bifunctional C-2 (5)- or C-3-substituted derivatives, i.e., regioisomeric sulfonyl halides, carboxylic acids, aldehydes and nitriles. Introduction of the CF₃ substituent into a position of the pyrrole ring provided efficient insecticides, i.e. chlorfenapyr, tralopyril, and potent biocide.

Aquino, E. da C., Leonel, G., et al. in The Journal of Organic Chemistry, (2015) 80 (24), 12453-12459 disclosed the Chemoselective Synthesis of 1-Substituted 4-Amino-2- (trifluoromethyl)-1H-pyrroles through the Heterocyclization Reaction of 4-Methoxy-5-bromo-1,1,1-trifluoropent-3-en-2-ones with Amines. It has disclosed a concise method to synthesize 1-substituted 4-amino-2-(trifluoromethyl)-1H-pyrroles from the heterocyclization reaction of 5-bromo-4-methoxy-1,1,1-trifluoropent-3-en2-ones with amines.

Despite plethora of numerous methods and processes reported in the prior art for synthesis of chlorfenapyr, there are still several number of drawbacks existing in process being utilized commercially including resulting in the low quality of the chlorfenapyr crude product, long reaction time, hazardous raw materials use and high production cost.

In addition, these synthetic routes also have the problems of poor reaction selectivity and low reaction yield. Furthermore, a large amount of water is wasted in subsequent filtrations during workup of reaction in order to scrub the additional byproducts and key process related impurities. All Chlorfenapyr methods have been reported with use of solvents like dimethylformamide (DMF), POCl₃ (Vilsmeier reagent) or PCl₃ and Et3N. These reactions are known to be highly exothermic and require lot of temperature control on commercial production level besides producing lot of color insoluble matter and poor yields, quality.

Therefore, still there exists a need of an economical, time saving and environment friendly method of preparation of Chlorfenapyr with high yield and quality that not only alleviates the above mentioned drawbacks in the purview of cited prior art but also provides safe an plant level amenable process as economically viable solution.

### Object of the Invention

The main object of the present invention is to provide a commercially viable and industrially up scalable method of synthesis of chlorfenapyr from tralopyril and diethoxymethane.

Another object of the present invention is to provide a method of synthesis of chlorfenapyr using a halogenating agent as safer reagent and in the presence of a base.

Yet another object of the present invention is to provide a method of synthesis of Chlorfenapyr with high yield (exceeding 90% w/w) and purity of exceeding 98% w/w.

Still another object of the present invention is to provide a method of synthesis of chlorfenapyr that is commercially viable, requiring less reaction time and which is very much suitable for manufacturing of chlorfenapyr in a large-scale production industrially.

### SUMMARY OF THE INVENTION

The present invention relates to a easy and thermally controllable and commercially viable method of preparation of a halogenated pyrrole.

More particularly, the present invention relates to the method of synthesis of Chlorfenapyr (a halogenated pyrrole) from tralopyril and ethylal in presence of a halogenating agent as thionyl chloride in the presence of organic base.

In one of the embodiment, the present invention relates to a process preparing Chlorfenapyr, comprising the steps of-
a.) reacting tralopyril (or 4-bromo-2-(4-chlorophenyl)-5-(trifluoromethyl)-1H-pyrrole-3-carbonitrile) and ethylal (or diethoxymethane) in presence of hydrocarbon solvent at temperature ranging between 20-30° C.;
b.) chlorinating the step a) solution with chlorinating agent selected as thionyl chloride at a temperature ranging between 15-80° C.;
c.) adding an organic base slowly at a temperature ranging between 15-80° C.;
d.) isolating highly pure Chlorfenapyr.

In another embodiment, the present invention relates to a process wherein step of isolating highly pure Chlorfenapyr comprising the further steps of-
i). reaction mass is allowed to cool down to room temperature;
ii). reaction is terminated with water addition to make biphasic mixture;
iii.) stir the biphasic mixture for 10-60 minutes followed by separating the layers;
iv.) separated aqueous layer is again washed with toluene;
v.) combine the organic layers and washed with water;
vi.) solvent is from organic layer is distilled off;
vii) adding aqueous methanol (using aqueous methanol composition ranging between (10-40% v/v) followed by stirring for 30-120 mins;
viii) isolate the highly pure Chlorfenapyr by filtration;
ix) drying the filtered material under vacuum at 40-70° C.

In yet another embodiment, the present invention relates to highly pure Chlorfenapyr obtained by using thionyl chloride as chlorinating agent, having purity exceeding 98% w/w.

According to the present invention, the pyrrole substituted aromatic hydrocarbon and the hydrocarbon solvent are used as starting materials of the reaction whereby the pyrrole is 4-bromo-2-(4-chlorophenyl)-5-(trifluoromethyl) pyrrole-3-carbonitrile or tralopyril; the hydrocarbon solvent is selected as toluene; and the alkylating agent is diethoxymethane (Ethylal). The halogenating agent used in said invention Chlorfenapyr process is thionyl chloride and the base is organic based selected as triethylamine.

The above objects and advantages of the present invention will become apparent from the hereinafter set forth detailed description of the invention provided herewith.

### DESCRIPTION OF THE INVENTION

The present invention will now be described hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

The present invention now will be described hereinafter with reference to the detailed description, in which some, but not all embodiments of the invention are indicated. Indeed, the invention may be embodied in many different forms and should not be construed as limited to the embodiments set forth herein; rather, these embodiments are provided so that this disclosure will satisfy applicable legal requirements. Like numbers refer to like elements throughout.

The present invention is described fully herein with non-limiting embodiments and exemplary experimentation.

The present invention provides a thermally controllable and commercially viable method of preparation of a halogenated pyrrole. More particularly, the present invention relates to the method of synthesis of Chlorfenapyr from tralopyril and ethylal in presence of a halogenating agent i.e. thionyl chloride.

The method of synthesis of Chlorfenapyr according to the present invention is demonstrated through the following scheme in more detail:

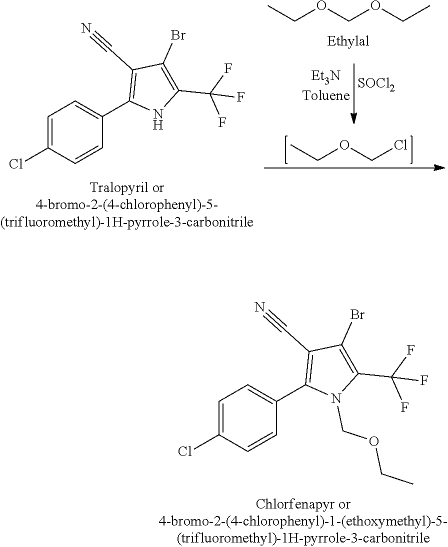

In one of the embodiment, the present invention provides—a process preparing Chlorfenapyr, comprising the steps of-
a.) reacting tralopyril (or 4-bromo-2-(4-chlorophenyl)-5-(trifluoromethyl)-1H-pyrrole-3-carbonitrile) and ethylal (or diethoxymethane) in presence of hydrocarbon solvent at temperature ranging between 20-30° C.;
b.) chlorinating the step a) solution with chlorinating agent selected as thionyl chloride at a temperature ranging between 15-80° C.;
c.) adding an organic base slowly at a temperature ranging between 15-80° C.;
d.) isolating highly pure Chlorfenapyr.

This process preparing Chlorfenapyr according to the present invention utilizes reactants used in the step a.) in molar ratios ranging between-tralopyril (or 4-bromo-2-(4-chlorophenyl)-5-(trifluoromethyl)-1H-pyrrole-3-carbonitrile): ethylal (or diethoxymethane) 1; 1-1.5.

The embodied process according to the present invention for preparing Chlorfenapyr wherein reactants used in the step b) and step c) are used in molar ratios with respect to 1 mole of tralopyril (or 4-bromo-2-(4-chlorophenyl)-5-(trifluoromethyl)-1H-pyrrole-3-carbonitrile) ranging between-thionylchloride and triethylamine 1-1.1:1.2-1.8

The hydrocarbon solvent used in the step a) may be selected from toluene, xylene, benzene, or cyclohexane or cyclopentane, however, in one of the preferred embodiment toluene was used as most economically viable solvent and with easy handling and recovering without any degradation expectations during the course of reaction. Inventor of the present invention surprisingly found that such hydrocarbon solvent makes reaction not only easily amenable to scale up but also devoid of several process related impurities formation and ease in handling compared from several reported processes in the literature.

The organic base used in the process is selected from triethyl amine, trimethyl amine, triisopropyl amine or diisobutyl methyl amine, however, in one of the preferred embodiment triethyl amine was used as most economically viable and abundantly available organic base.

The process for preparing Chlorfenapyr as per present invention wherein organic base used in the process was added at temperature ranging between 50-80° C. with slow addition and controlled course of reaction.

The process preparing Chlorfenapyr according to present invention in another embodiment provides the process for isolating highly pure Chlorfenapyr, which is comprising the steps of-
i). reaction mass after reaction completion was allowed to cool down to room temperature;
ii). reaction is terminated with water addition to make biphasic mixture;
iii.) stir the biphasic mixture for 10-60 minutes followed by separating the layers;
iv.) separated aqueous layer is again washed with toluene;
v.) combine the organic layers and washed with water;
vi.) solvent is from organic layer is distilled off;
vii) add aqueous methanol followed by stirring for 30-120 mins;
viii) isolate the highly pure Chlorfenapyr by filtration;
ix) drying the filtered material under vacuum at 40-70° C.

The above process according to the embodiment for isolating highly pure Chlorfenapyr wherein step vii) of adding aqueous methanol comprise using aqueous methanol composition ranging between 10-40% v/v. The surprising observation according to the scientist provided that this range is more appropriate for not compromising on yields and efficiency as well as retaining high quality of product consistently in continuous running batches. Several failure experiments of poor yield were observed for exceeding methanol quantity in the composition viz, using more than 40% methanol has resulted in poor yield and recoveries.

The present invention embodiment provided a process of preparing Chlorfenapyr possessing highly pure Chlorfenapyr having yields exceeding 90% w/w and purity exceeding 98% w/w.

A highly pure Chlorfenapyr obtained by using specifically thionyl chloride as chlorinating agent was found to continuously yielding high purity of exceeding 98% w/w.

The plethora of literature reported for preparing Chlorfenapyr reported the chlorination reaction with chlorinating agent used as PCl₃ (Phosphorus trichloride) or POCl₃ (Phosphorus oxy chloride) using Et₃N (triethylamine) in DMF+toluene or toluene or the like. Inventors of the present invention have miserably suffered setbacks using these chlorinating agents resulting in often yellow colored material and involving further purifications and generating environmental hazards of handling additional pollution load.

When inventors surprisingly carried out the same reaction with thionyl chloride, Et₃N in toluene, it has excellent course of reaction and high yield, high quality and besides advantage of exceptional less reaction time compared to the PCl₃ and POCl₃ reactions.

According to the scientists of the present invention, it was also surprising that the reaction was quite less exothermic and temperature is in well control compared to the PCl₃ and POCl₃.

Insoluble materials generated in reactions performed using PCl₃ and POCl₃ needed to do more number of filtrations, washings etc during the workup operations, where as in thionyl chloride reactions only gases are formed which are scrubbed and controlled into quenching water. Lot of colored materials formed in the PCl₃ and POCl₃ reactions and its handling became a concern to environment while disposing. Further, the price of thionyl chloride was found quite less compared to PCl₃ and POCl₃. Also handling of thionyl chloride was observed easy in the commercial scales, hence process is commercially viable.

In a preferred embodiment, the present invention provides a method for synthesis of chlorfenapyr, comprising the steps of charging tralopyril and organic solvent as toluene in a reactor at room temperature and stirred for a period of 10 minutes; charging the diethoxymethane (or Ethylal) in said reactor at room temperature to obtain a mixture; heating the said mixture at a temperature ranging from 15-80° C. followed by continuous controlled addition of thionyl chloride at temperature ranging between 15-80° C. for one hour to obtain a resulting mixture.

It is maintained continuous stirring for the resulting mixture at temperature ranging between 15-80° C. for another one hour; adding organic base as triethylamine at temperature ranging between 15-80° C. It may be added within one hour continuously maintaining the temperature within range, however, time duration may exceed or less depending upon the temperature control measures utilized.

The reaction mixture so obtained is further maintained for one-two hours in the same temperature range and allowed said reaction mixture to settle at room temperature with confirmation of the absence of tralopyril by TLC or HPLC, whichever is appropriately used for preliminary confirmation.

For this organic phase toluene based reaction, adding demineralized water to obtain a biphasic mixture under continuous stirring for 30-60 minutes.

The aqueous and organic layers are separated and separated aqueous layer is washed with organic hydrocarbon solvent-toluene.

Collecting all organic layer together and washing said organic layers with a mixture of water and 1-8% of aqueous sodium bicarbonate solution.

The clean organic layer is subjected for distillation to obtain a crude material followed by adding 5-40% aqueous methanol to said crude material and stirring for 30-120 minutes at room temperature and filtering the same to obtain a solid crystalline material cake. The filtered cake was washed using of 5-30% aqueous methanol and drying in vacuum at 60° C. to obtain highly pure Chlorfenapyr.

The chlorfenapyr produced by said method possess purity of ≥98% w/w and is yielded in amount of 109.6 g or 94%. The molar ratio of the starting materials, that is, diethoxymethane, thionyl chloride and triethylamine is 1:1.1:1.1:1.4.

### EXAMPLES

Below scheme for Chlorfenapyr synthesis is followed as the best mode illustration for the process according to the present invention.

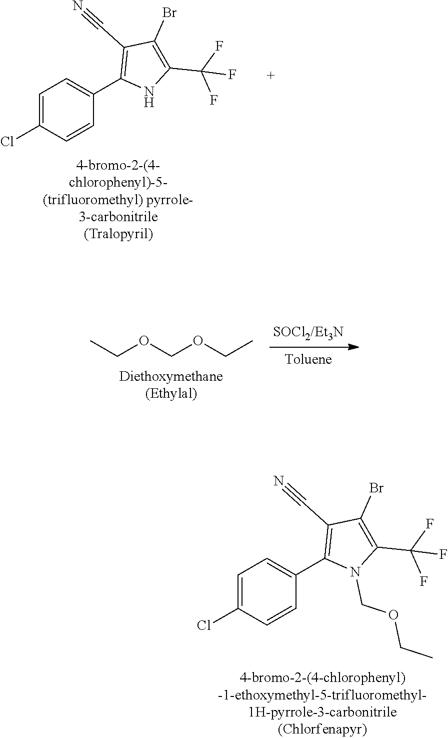

### Scheme: For Chlorfenapyr Synthesis as Per Present Invention

### Example-1: Chlorfenapyr Synthesis Using Thionyl Chloride as Chlorinating Agent (Mole Ratio 1.1 w.r.t Tralopyril and Added at Temp 60-70° C.)

Tralopyril or 4-bromo-2-(4-chlorophenyl)-5-(trifluoromethyl) pyrrole-3-carbonitrile (100 g, 0.286 moles) is charged in toluene (300 mL) and stirred for 10 minutes at room temperature. Diethoxymethane (32.77 g, 0.314 moles) is charged in to the reactor at room temperature. Resulting mixture is heated to 60° C. Thionyl chloride (37.44 g, 0.314 moles) is added at 60-70° C. for 30 minutes. Resulting mixture is stirred for another 1 hour at 70° C. Triethyl amine (40.49 g, 0.40 moles) is added for 1-2 hour at 60-70° C. Reaction mixture is maintained for 1-2 hours at 60-70° C. Reaction completion is monitored by thin layer chromatography/HPLC. After absence of starting material, reaction mass is allowed to room temperature and terminated with water (200 mL). Biphasic mixture is stirred for 30 minutes and separated layers. Aqueous layer is again washed with toluene (100 mL). The combined organic layers are washed with 200 ml water. Solvent is removed from organic layer by distillation. 20% aqueous methanol (200 mL) is added to the crude material, stirred for 1 hour at RT and filtered through Buchner flask. The filter cake is washed with 20% aqueous methanol (50 mL) and dried in vacuo at 60 oC to give Chlorfenapyr (109.6 g, yield: 94%). Its purity analysed by HPLC as (≥98% w/w).

In the above method, the molar ratio of starting material, diethoxymethane, thionylchloride and triethylamine is 1:1.1:1.1:1.4,

### Example-2: Chlorfenapyr Synthesis Using Thionyl Chloride as Chlorinating Agent (Mole Ratio 1.1 w.r.t Tralopyril and Added at Room Temperature Demonstrating Safety of Reaction)

4-bromo-2-(4-chlorophenyl)-5-(trifluoromethyl) pyrrole-3-carbonitrile (100 g, 0.286 moles) is charged in toluene (300 mL) and stirred for 10 minutes at room temperature. Diethoxymethane (32.77 g, 0.314 moles) is charged in to the reactor at room temperature. Resulting mixture is heated to 60° C. Thionyl chloride (37.44 g, 0.314 moles) is added at room temperature for 30 minutes. Resulting mixture is stirred for another 1 hour at 50-55° C. Triethyl amine (40.49 g, 0.40 moles) is added for 1-2 hour at 50-55° C. Reaction mixture is maintained for 1-2 hours at same temperature. Reaction completion is monitored by thin layer chromatography/HPLC. After absence of starting material, reaction mass is allowed to room temperature and terminated with water (200 mL). Biphasic mixture is stirred for 30 minutes and separated layers. Aqueous layer is again washed with toluene (100 mL). The combined organic layers are washed with 200 ml water. Solvent is removed from organic layer by distillation.

20% aqueous methanol (200 mL) is added to the crude material, stirred for 1 hour at RT and filtered through Buchner flask. The filter cake is washed with 20% aqueous methanol (50 mL) and dried in vacuum at 60° C. to give Chlorfenapyr (108.43 g, yield: 93%). Its purity analysed by HPLC as (≥98% w/w).

In the above method, the molar ratio of starting material, diethoxymethane, thionylchloride and triethylamine is 1:1.1:1.1:1.4.

### Example-3: Chlorfenapyr Synthesis Using Thionyl Chloride as Chlorinating Agent (Mole Ratio 1.1 w.r.t Tralopyril and Added at Temp 60-70° C. and Organic Base 1.78 Mole)

4-bromo-2-(4-chlorophenyl)-5-(trifluoromethyl) pyrrole-3-carbonitrile (100 g, 0.286 moles) is charged in toluene (300 mL) and stirred for 10 minutes at room temperature. Diethoxymethane (41.70 g, 0.40 moles) is charged in to the reactor at room temperature. Resulting mixture is heated to 60° C. Thionyl chloride (37.44 g, 0.314 moles) is added at 60-70° C. for 30 minutes. Resulting mixture is stirred for another 1.0 hour at 70° C. Triethyl amine (52.046 g, 0.51 moles) is added for 1-2 hour at 60-70° C. Reaction mixture is maintained for 1-2 hours at 60-70° C. Reaction completion is monitored by thin layer chromatography/HPLC. After absence of starting material, reaction mass is allowed to room temperature and terminated with water (200 mL). Biphasic mixture is stirred for 30 minutes and separated layers. Aqueous layer is again washed with toluene (100 mL). The combined organic layers are washed with 200 ml water. Solvent is removed from organic layer by distillation.

20% aqueous methanol (200 mL) is added to the crude material, stirred for 1 hour at RT and filtered through Buchner flask. The filter cake is washed with 20% aqueous methanol (50 mL) and dried in vacuum at 60° C. to give Chlorfenapyr (110.78 g, yield: 95%). Its purity analysed by HPLC as (≥98% w/w).

In the above method, the molar ratio of starting material, diethoxymethane, thionylchloride and triethylamine is 1:1.4:1.1:1.8.

### Example-4: Chlorfenapyr Synthesis Using Thionyl Chloride as Chlorinating Agent (Mole Ratio 1.1 w.r.t Tralopyril and Added at Temp 60-70° C.)

4-bromo-2-(4-chlorophenyl)-5-(trifluoromethyl) pyrrole-3-carbonitrile or Tralopyril (100 g, 0.286 moles) is charged in toluene (300 mL) and stirred for 10 minutes at room temperature. Diethoxymethane (41.70 g, 0.40 moles) is charged in to the reactor at room temperature. Resulting mixture is heated to 60° C. Thionyl chloride (37.44 g, 0.314 moles) is added at 60-70° C. for 30 minutes. Resulting mixture is stirred for another 1.0 hour at 70° C. Triethyl amine (52.046 g, 0.51 moles) is added for 1-2 hour at 60-70° C. Reaction mixture is maintained for 1-2 hours at 60-70° C. Reaction completion is monitored by thin layer chromatography/HPLC. After absence of starting material, reaction mass is allowed to room temperature and terminated with water (200 mL). Biphasic mixture is stirred for 30 minutes and separated layers. Aqueous layer is again washed with toluene (100 mL). The combined organic layers are washed with 200 ml water. Solvent is removed from organic layer by distillation.

20% aqueous methanol (200 mL) is added to the crude material, stirred for 1 hour at RT and filtered through Buchner flask. The filter cake is washed with 20% aqueous methanol (50 mL) and dried in vacuo at 60 oC to give Chlorfenapyr (110.78 g, yield: 95%). Its purity analysed by HPLC as (≥98% w/w).

In the above method, the molar ratio of starting material, diethoxymethane, thionylchloride and triethylamine is 1:1.4:1.1:1.8.

In the present invention, inventors utilized the tralopyril, prepared using below process (GIVEN HEREINBELOW REFERENCE EXAMPLES) known in the open source literature, though the process still needs several improvement in tralopyril process.

### Reference Examples for Synthesis of Tralopyril

Synthesis of tralopyril is a three step synthesis. Individual steps are detailed herein below.

### Step-I: Preparation of 4-(4-chlorophenyl)-2-(trifluoromethyl)oxazol-5 (2H)-One

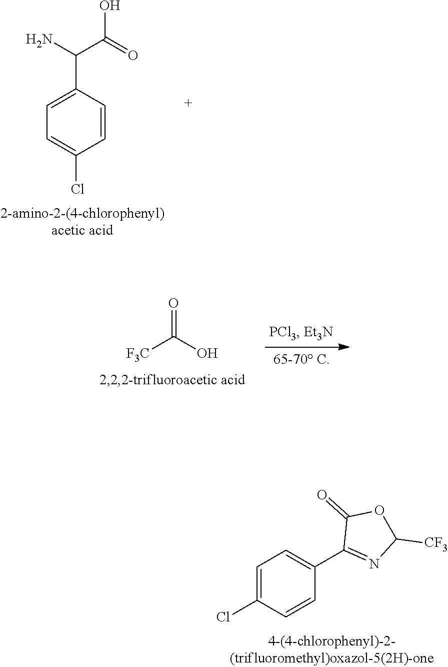 [SMILES: NC(C(=O)O)c1ccc(Cl)cc1 ; O=C(O)C(F)(F)F ; O=C1OC(C(F)(F)F)N=C1c1ccc(Cl)cc1]

2-Amino-4′-chloro phenylacetic acid (4-chlorophenylglycine) (100 g, 0.538 moles) is charged in Acetonitrile (300ml) 25-30° C. The reaction mass is cooled 15-20° C. Trifluoroacetic acid (67.5 g, 0.592 moles) is added for 30 minutes at 15-20° C. Triethyl amine (65.42 g, 0.646 moles) is added at same temperature for 30 minutes. Phosphorus trichloride (73.88, 0.431 moles) is added for 1 hour at same temperature. The reaction mass is allowed to heat up to 30° C. and slowly increases to 60-65° C. and maintained 8 hours at same temperature. The reaction completion is monitored by TLC. After completion of the reaction, acetonitrile is removed under reduced pressure. Toluene (300 ml) is added to the mass and stirred for 15 minutes. Reaction mass is quenched with water (300 ml), layers are separated. Solvent is removed under pressure to get the yellow liquid compound (118 g, Purity: 95% by HPLC area and yield 83% w/w).

### Step-2: Preparation of 2-(4-chlorophenyl)-5-(trifluoromethyl)-1H-pyrrole-3-carbonitrile

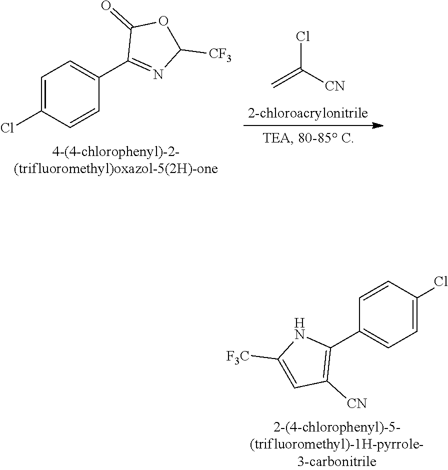

4-(4-chlorophenyl)-2-(trifluoromethyl)oxazol-5 (2H)-one (118 g, 0.44 moles) is charged in DMF (150ml) at 25-30° C. Sodium carbonate (46.2 g, 0.528 moles) is added over period of 15 minutes at 25-30° C. The reaction mass is stirred for 15 minutes at 25-30° C. 2-Chloroacrylonitrile is added to the reaction mass at 25-30° C. for over period of 2-3 hours. Reaction is monitored by HPLC. After completion of the reaction, reaction mass is subjected to filtration. DMF is removed from filtrate by using reduced pressure. The reaction mass is purified with aqueous methanol to get the cream compound (100 g, yield 82%)

### Step-III: Preparation of 4-bromo-2-(4-chlorophenyl)-5-(trifluoromethyl)-1H-pyrrole-3-carbonitrile (tralopyril)

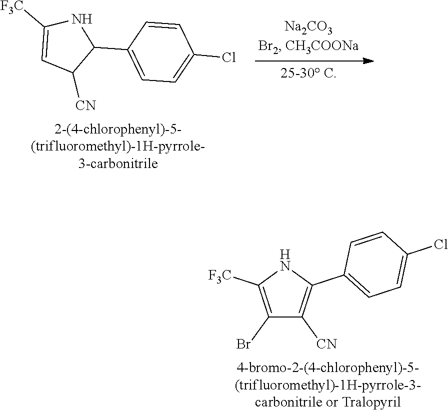 [SMILES: N#CC1C=C(C(F)(F)F)NC1c1ccc(Cl)cc1 ; N#Cc1c(-c2ccc(Cl)cc2)[nH]c(C(F)(F)F)c1Br]

2-(4-chlorophenyl)-5-(trifluoromethyl)-1H-pyrrole-3-carbonitrile (100 g, 0.369 moles) is charged at 25-30° C. in methanol (200 ml). The reaction mass stirred for 10-15 minutes. Sodium acetate (30.3 g, 369 moles) is charged at 25-30° C. The reaction mass is stirred for 15 minutes. Bromine (58.9 g, 0.369 moles) is charged very slowly at 25-30° C. over a period of 1 hour. Reaction is monitored by HPLC. After completion of reaction, reaction mass is quenched with sodium bisulphite solution and the cream-coloured precipitate is formed in the reaction mass. This reaction mass is further stirred for another 1 hour at 25-30° C. The reaction mass is filtered and washed with water to get the 4-bromo-2-(4-chlorophenyl)-5-(trifluoromethyl)-1H-pyrrole-3-carbonitrile (tralopyril, 120 g, yield 93%).

While the foregoing pages provide a detailed description of the preferred embodiments of the invention, it is to be understood that the description and examples are for illustrative purpose only of the principles of the invention and not limiting to its scope. Furthermore, as many changes can be made to the invention without departing from the scope of the invention, it is intended that all material contained herein be interpreted as illustrative of the invention and not in a limiting sense.

We claim:

1. The process preparing Chlorfenapyr, comprising the steps of-
a). reacting tralopyril (or 4-bromo-2-(4-chlorophenyl)-5-(trifluoromethyl)-1H-pyrrole-3-carbonitrile) and ethylal (or diethoxymethane) in presence of hydrocarbon solvent at temperature ranging between 20-30° C.;
b). chlorinating the step a) solution with chlorinating agent selected as thionyl chloride at a temperature ranging between 15-80° C.;
c). adding an organic base slowly at a temperature ranging between 15-80° C.;
d). isolating highly pure Chlorfenapyr.

2. The process preparing Chlorfenapyr according to claim 1, wherein reactants used in the step
a) are used in molar ratios ranging between—
tralopyril (or 4-bromo-2-(4-chlorophenyl)-5-(trifluoromethyl)-1H-pyrrole-3-carbonitrile):
ethylal (or diethoxymethane) 1:1-1.5.

3. The process preparing Chlorfenapyr according to claim 1, wherein reactants used in the step b) and step c) are used in molar ratios with respect to 1 Mole of tralopyril (or 4-bromo-2-(4- chlorophenyl)-5-(trifluoromethyl)-1H-pyrrole-3-carbonitrile) ranging between—thionylchloride and triethylamine 1-1.1:1.2-1.8.

4. The process preparing Chlorfenapyr according to claim 1, wherein hydrocarbon solvent used in the step a) is selected from toluene, xylene, benzene, or cyclohexane or cyclopentane.

5. The process preparing Chlorfenapyr according to claim 1, wherein organic base used in the step c) is selected from triethyl amine, trimethyl amine, triisopropyl amine or diisobutyl methyl amine.

6. The process preparing Chlorfenapyr according to claim 1, wherein organic base used in the step c) is added at temperature ranging between 50-80° C.

7. The process preparing Chlorfenapyr according to claim 1, wherein step d) of isolating highly pure Chlorfenapyr comprising the further steps of-
i). reaction mass is allowed to cool down to room temperature;
ii). reaction is terminated with water addition to make biphasic mixture;
iii.) stir the biphasic mixture for 10-60 minutes followed by separating the layers;
iv.) separated aqueous layer is again washed with toluene;
v.) combine the organic layers and washed with water;
vi.) solvent is from organic layer is distilled off;
vii) add aqueous methanol followed by stirring for 30-120 mins;
viii) isolate the highly pure Chlorfenapyr by filtration;
ix) drying the filtered material under vacuum at 40-70° C.

8. The process preparing Chlorfenapyr according to claim 7, wherein step vii) of adding aqueous methanol comprise using aqueous methanol composition ranging between 10-40% v/v.

9. The process of preparing Chlorfenapyr according to claim 1, wherein highly pure Chlorfenapyr obtained in the step d) having yield exceeding 90% w/w and purity exceeding 98% w/w.

10. Highly pure Chlorfenapyr obtained by using thionyl chloride as chlorinating agent, having purity exceeding 98% w/w.
